(19) United States
(12) Patent Application Publication
(10) Pub. No.: US 2020/0232982 A1
(43) Pub. Date: Jul. 23, 2020

(54) DIAGNOSTIC SYSTEM

(71) Applicant: Ellume Limited, East Brisbane, Queensland (AU)

(72) Inventors: Sean Andrew Parsons, East Brisbane (AU); Scott Robert Fry, East Brisbane (AU); Andrea Ranzoni, East Brisbane (AU)

(21) Appl. No.: 16/486,819

(22) PCT Filed: Feb. 21, 2018
(86) PCT No.: PCT/AU2018/050140

§ 371 Date: Aug. 16, 2019

(30) Foreign Application Priority Data
Feb. 21, 2017 (AU) 2017900569

(51) Int. Cl. G01N 33/543; G01N 33/53; G16H 50/30; G16H 50/20

(52) U.S. Cl. CPC G01N 33/54386; G16H 50/20; G16H 50/30; G01N 33/5302

(57) ABSTRACT

In some embodiments a diagnostic system is disclosed including a test device for performing diagnostic analysis of a biological sample from a human or animal body, the test device having an assay and a reader to analyse the sample and determine one or more biological conditions and to generate a result data signal containing information on the determination of the one or more biological conditions. The system also comprises a base device to releasably receive the test device, receive the result data signal from the test device, and display results of testing based on the received result data signal. In some embodiments a fluid receptacle is configured to be releasably retained to a test device, the test device comprising a housing, the housing having an engagement element for releasably retaining the fluid receptacle on an outer surface of the housing.

### CROSS-REFERENCE TO RELATED APPLICATIONS

The present application claims priority to Australian provisional application no. 2017900569 filed 21 Feb. 2017, the entire content of which is herein incorporated by reference.

### TECHNICAL FIELD

The present invention relates to a diagnostic system as well as a method of performing diagnostic analysis of a biological sample.

### BACKGROUND

There exist many types of diagnostic devices for identifying target biological entities and therefore target medical conditions in a person or animal. The devices analyse a biological sample provided by the person or animal, such as a urine sample, blood sample or otherwise, and identify a biological entity in the sample that provides a marker for the target condition. Increasingly, the diagnostic devices are being designed for point-of-care testing, which brings the diagnostic tests conveniently to the patient or caregiver.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

In some aspects of the present disclosure, a diagnostic system is disclosed including: at least one test device for performing diagnostic analysis of a biological sample from a human or animal body, the test device configured to analyse the sample and determine one or more biological conditions and to generate a result data signal containing information on the determination of the one or more biological conditions; and a base device configured to receive the test device and receive the result data signal from the test device.

In one aspect of the present disclosure, there is provided a diagnostic system comprising:

at least one test device for performing diagnostic analysis of a biological sample from a human or animal body, the at least one test device comprising:
an assay unit configured to receive the biological sample for testing for the presence or absence of one or more biological entities in the biological sample; and
a reader configured to determine one or more biological conditions based on the presence or absence of the one or more biological entities at the assay unit, and to generate a result data signal containing information on at least the determination of the one or more biological conditions; and

a base device comprising a port for releasably receiving the test device; and a receiver for receiving the result data signal when the test device is received by the base device; the base device further comprising, or being configured to connect to, a display for displaying results of testing of the biological sample based on the received result data signal.

In some embodiments, the one or more biological entities may provide a marker for the one or more biological conditions. In this regard, the one or more biological conditions may comprise one or more of chlamydia, colorectal cancer, Helicobacter pylori infection, influenza, legionella, mononucleosis, ovulation phase in a menstrual phase, osteoporosis, pregnancy, prior subjection to myocardial infarction, renal failure, respiratory syncytial virus infection, pneumococcal infection, and streptococcal pharyngitis, for example.

The assay unit may comprise a test portion to test for the presence or absence of a biological entity in the biological sample. In some embodiments, the test portion may be an immunochromatography test strip, although other test portions may be used, and which rely on, for example, fluorescence technologies, absorbance/extinction technologies, electrochemical technologies and/or enzymatic amplification technologies i.e. signal amplification with reporter enzyme or template amplification for molecular detection.

The reader may comprise one or more light sources adapted to transmit excitation light to one or more portions of the test strip, and one or more light detectors capable of monitoring light reflection or light output at one or more portions of the test strip. The reader may comprise a processor that may be configured to make a determination about the presence or absence of the one or more biological entities in the biological sample based on the intensity of the light detected by the one or more light detectors.

The reader may comprise a non-transitory computer-readable memory medium comprising instructions that cause the processor to determine the one or more biological conditions and generate the result data signal.

The assay unit and the reader may be located within a housing of the test device. The housing of the test device may have an end that is adapted to be inserted into the port of the base device. A test device connector may be provided at or adjacent the end of the housing. Insertion of the end of the housing and/or the test device connector into the port of the base device may establish a connection between the test device and the base device to enable transfer of the result data signal to the base device. Establishment of the connection may be automatic.

In some embodiments, the base device may comprise an electronics unit that includes the port to receive the test device and specifically to electrically couple to the test device so as to allow for data transmission between the test device and the electronics unit. The electronics unit may comprise connectivity components for detecting a successful connection between the at least one test device and the electronics unit. In this regard, data transmission between the at least one test device and electronics unit may only commence after a successful connection has been registered by the electronics unit.

In some embodiments, that base device may comprise the display for displaying results of testing of the biological sample based on the received result data signal. In some embodiments, a display unit may be provided that comprises the display. In some embodiments, the base device may comprise the display unit. The display unit may be integrated with the electronics unit during manufacture or may be manufactured separately and may be connected to the electronics unit prior to use. For example, in some embodiments, the display unit may be provided by a computer device, such as a smartphone, personal digital assistant or tablet computer or a standalone television, monitor or otherwise. In some embodiments, the electronics unit may comprise a housing adapted to at least partially house the display unit. For example, in some embodiments, the electronics unit may substantially encase the display unit except, for example, for the display of the display unit. The electronics unit may comprise a window or other opening through which the display of the display unit can be observed.

In some embodiments, as indicated, the display unit may be integrated with the electronics unit during manufacture. In this regard, the entire base device may be provided by a computer device, such as a smartphone, personal digital assistant, tablet computer or otherwise.

In some embodiments, the electronics unit and the display unit may be positioned separately. A communications link may be provided between the electronics unit and the display unit. The communication link may be a wired link and/or a wireless link. For example, a cable may connect the electronics unit to the display unit. The electronics unit may be connected via the cable to a port of the display unit such as a USB port, miniUSB port, a serial port, an HDMI port, a VGA port, a DVI port, or other standard or non-standard communications port. Additionally or alternatively, the electronics unit may be connected to the display unit via Bluetooth, Wi-Fi, or other wireless link.

The electronics unit may comprise a display controller for controlling displaying of results by the display unit and/or the display unit may comprise a display controller for controlling displaying of results by the display unit.

The base device may be provided in the form of a hand-held device. In other embodiments, the base device may be provided by apparatus that rests on a desk or table. In some embodiments, the at least one test device may be a hand-held device. The at least one test device may also be disposable and configured for single-use only. In contrast to the test device, the base device may be configured for multiple use.

In some embodiments, the electronics unit may be configured to distribute power to the at least one test device and/or the display unit. A series of conditioning elements may be provided in the electronics unit to regulate the transmission of power to the test device and/or the display unit. In some embodiments, the at least one test device may have no integral power source, with all power, e.g. for the performing of the reading operations, being provided from the base device.

In some embodiments, the base device, e.g., the electronics unit of the base device, includes two or more ports, each of the two or more ports being configured to releasably receive a test device. In general, by providing reader elements in the test device, multiple test devices of the same type or different types can be connected simultaneously to the base device, without the base device requiring any bespoke reader elements and/or added functionality to perform the diagnostic testing. This reduces processing requirements of the base device itself.

In another aspect of the present disclosure, there is provided a test device for a base device having a port for releasably receiving the test device, the test device comprising:

an assay unit configured to receive a biological sample from a human or animal body for testing for the presence or absence of one or more biological entities in the biological sample; and

a reader configured to determine one or more biological conditions based on the presence or absence of the one or more biological entities at the assay unit, and to generate a result data signal containing information on at least the determination of the one or more biological conditions,

wherein the base device is configured to receive the result data signal when the test device is received by the base device for displaying results of testing of the biological sample based on the received result data signal.

In another aspect of the present disclosure, there is provided a base device, or an electronics unit of a base device, comprising one or more ports for releasably receiving one or more test devices, each test device being according to the immediately preceding aspect. The base device or electronics unit may include any one or more of the features of the base devices and electronics units as described above.

In a further aspect of the present disclosure, there is provided a method for performing diagnostic analysis of a biological sample from a human or animal body, the method comprising:

receiving the biological sample in a test device;

testing, using the test device, for the presence or absence of one or more biological entities in the biological sample;

determining, using the test device, one or more biological conditions based on the presence or absence of the one or more biological entities in the biological sample;

generating, at the test device, a result data signal containing information on at least the determination of the one or more biological conditions;

connecting the test device to a port of the base device;

receiving the result data signal by a receiver of the base device when the test device is connected to the base device; and

displaying results of testing of the biological sample based on the received result data signal.

The connecting of the test device to the port of the base device may be carried out prior to the generating of the result data signal or after generating of the result data signal. When connected prior to generating of the result data signal, the method may comprise receiving power at the test device from the base device to carry out the testing, determining and/or generating steps at the test device.

In yet a further aspect of the present disclosure, there is provided a test device for testing for the presence or absence of one or more biological entities in a biological sample, the test device comprising a housing, the housing having an engagement element for releasably retaining a fluid receptacle on an outer surface of the housing.

In yet a further aspect of the present disclosure, there is provided a fluid receptacle configured to be releasably retained to a test device, the test device comprising a housing, the housing having an engagement element for releasably retaining the fluid receptacle on an outer surface of the housing.

In the above aspects, the engagement element may be a recess adapted to receive a first end of the fluid receptacle. The engagement element may be adapted to receive the fluid receptacle in a releasably-locked manner In one embodiment, the engagement element and the end of the fluid receptacle have bayonet fittings for releasably locking to each other.

The fluid receptacle may provide a mixing chamber for mixing of a biological sample with a fluid such as a buffer solution. At a second end of the fluid receptacle, a cap may be provided that is releasably securable to the fluid receptacle, over an opening, e.g. via a screwthread. Fluid may be deposited into the fluid receptacle via the opening and the cap may be secured thereon, e.g. during manufacture or subsequently.

In use, the receptacle, e.g. containing buffer solution, may be locked to the test device and the cap subsequently released. A sample swab, or other sample-holding device, may be inserted into the receptacle, via the opening, and optionally stirred within the receptacle, enabling mixing of the sample with the buffer solution. Once mixing is complete, the cap may be replaced on the receptacle. After mixing, the mixture of sample and buffer solution may be held in the receptacle and/or on the sample swab. The mixture may be applied from any one of these items to another portion of the test device, e.g. to a sample port of the test device.

By providing an engagement element for supporting and retaining the fluid receptacle, use of the test device, e.g. in conjunction with a base device as described in preceding aspects, may be more straightforward. For example, a user may support the test device, or the base device connected to the test device, in one hand with the receptacle projecting from the test device. Meanwhile, with their other hand, the user may remove the cap of the receptacle, place the swab in the receptacle, stir the swab, replace the cap and apply the mixture to a sampling portion of the test device.

In some embodiments, to release the cap, the cap may be rotated in a direction, e.g. an anticlockwise direction, corresponding to a direction of rotation that may be used to lock the receptacle to the test device, ensuring that release of the cap does not cause unintentional release of the receptacle from the test device.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the invention will now be described, by way of example only, with reference to the accompanying drawings, in which:

FIG. 1 is a front view of a diagnostic system according to an embodiment of the present disclosure;

FIG. 2 is a schematic illustration of the diagnostic system of FIG. 1;

FIG. 3 is an isometric view of a test device of the diagnostic system of FIG. 1;

FIG. 3a is an exploded view of the test device of FIG. 3;

FIG. 4 is a schematic representation of a test strip used in the test device of FIG. 3, the schematic representation showing several regions that are arranged sequentially along the length of the test strip;

FIG. 5 is an isometric view of a buffer bottle that may be used in conjunction with the test device of FIG. 3;

FIG. 6 is a top isometric view of a base device of the diagnostic system of FIG. 1;

FIG. 7 is a bottom isometric view of the base device of FIG. 6;

FIG. 8 is another bottom isometric view of the base device of FIG. 6, without the outer casing;

FIG. 9 is a side view of the base device of FIG. 6;

FIG. 10 is an isometric view of a diagnostic system according to another embodiment of the present disclosure;

FIG. 11 is a flow diagram showing an example method of performing diagnostic analysis of a biological sample from a human or animal body;

FIG. 12a is an isometric view of a diagnostic system according to another embodiment of the present disclosure;

FIG. 12b is an isometric view of a diagnostic system according to yet another embodiment of the present disclosure

FIG. 13a is an isometric view of a diagnostic system according to another embodiment of the present disclosure; and

FIG. 13b is an isometric view of a diagnostic system according to yet another embodiment of the present disclosure.

### DESCRIPTION OF EMBODIMENTS

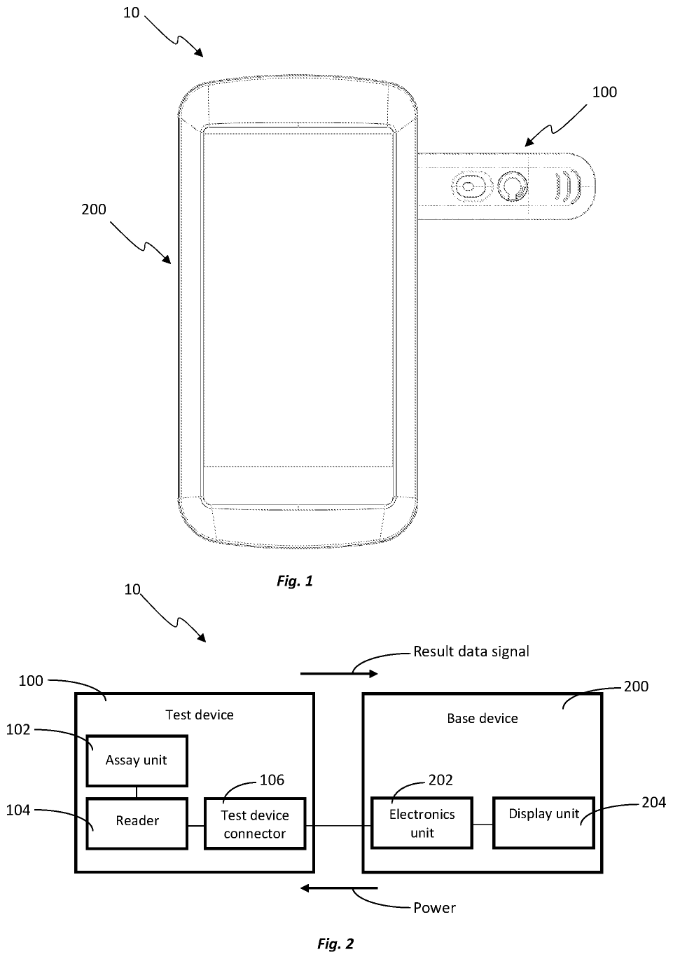

FIGS. 1 and 2 show a diagnostic system 10 according to an embodiment of the present disclosure. The diagnostic system 10 comprises at least one test device 100 and a base device 200 for use with the at least one test device 100.

As illustrated schematically in FIG. 2, each test device 100 includes an assay unit 102 configured to receive a biological sample from a human or animal body for testing for the presence or absence of one or more biological entities in the biological sample. Each test device 100 also includes a reader 104 including a processor and software capable of performing diagnostic analysis. The processor is configured to make a determination on whether a person providing the sample has, or does not have, one or more biological conditions based on the presence or absence of the one or more biological entities in the sample. In this regard, the present disclosure recognises that the biological entities can provide a marker for the biological condition(s).

In some embodiments, the one or more biological conditions may comprise, for example, one or more of chlamydia, colorectal cancer, Helicobacter pylori infection, influenza, legionella, mononucleosis, ovulation phase in a menstrual phase, osteoporosis, pregnancy, prior subjection to myocardial infarction, renal failure, respiratory syncytial virus infection, pneumococcal infection, and streptococcal pharyngitis.

The processor of the reader 104 is also configured to generate a result data signal containing information about the determination of the one or more biological conditions. The result data signal may transmitted from the test device 100 to the base device 200 via a test device connector 106. The base device 200 has an electronics unit 202 configured to receive the data signal from each test device 100, via electrical connection of the electronics unit 202 with the test device connector 106, and a display unit 204 for displaying the results of testing. In the present embodiment, diagnostic analysis of the sample is carried out solely by each test device 100, which significantly reduces processing requirements of the base device 200 of the present disclosure. The base device 200 may therefore act only as a means for displaying the results of testing and optionally for distributing power to the test device. This also allows for multiple test devices 100 to be connected simultaneously to the base device 200 without requiring any bespoke reader elements in the base device 200. The diagnostic system 10 may be differentiated in this regard from other systems that may employ an electronic reader in a base device to perform the diagnostic analysis of the sample rather than in the test device itself.

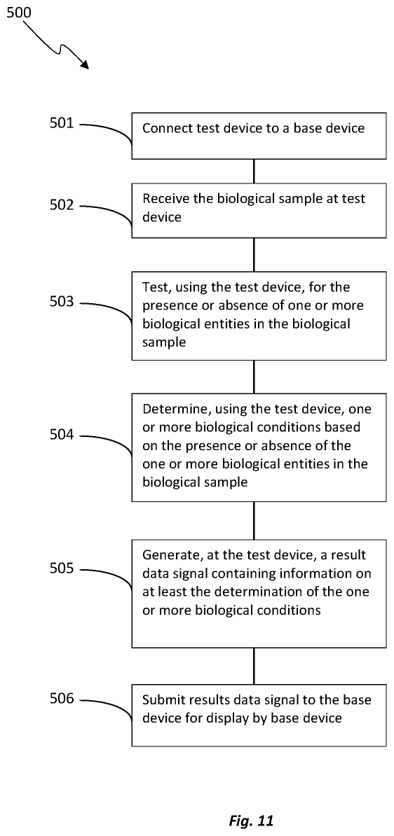

Following from this, in one embodiment, with reference to FIG. 11, a method 500 is carried out including, at 501, connecting the test device 100 to the base device 200; at 502, receiving the biological sample at the test device 100; at 503, testing, using the test device 100, for the presence or absence of one or more biological entities in the biological sample; at 504, determining, using the test device 100, one or more biological conditions based on the presence or absence of the one or more biological entities in the biological sample; at 505, generating, at the test device 100, a result data signal containing information on at least the determination of the one or more biological conditions; and, at 506, submitting the result data signal to the base device 200 for display of results by the base device 200. In some embodiments, depending on the reliance of the test device 100 on receiving power from the base device 200, the connecting of the test device 100 to the base device 200 may be carried out later in the method, e.g. at any time after receiving the biological sample and before submitting of the result data signal to the base device 200.

The assay unit 102 and reader 104 comprise components to test the biological sample. The specific components can depend on the type of test to be conducted. The reader 104 and assay unit 102 may be configured to test for the presence or absence of a single biological entity or multiple biological entities in the biological sample. Different detection technologies may be used within the test device 100, such as for example, fluorescence technologies, absorbance/extinction technologies, electrochemical technologies and/or enzymatic amplification technologies i.e. signal amplification with reporter enzyme or template amplification for molecular detection.

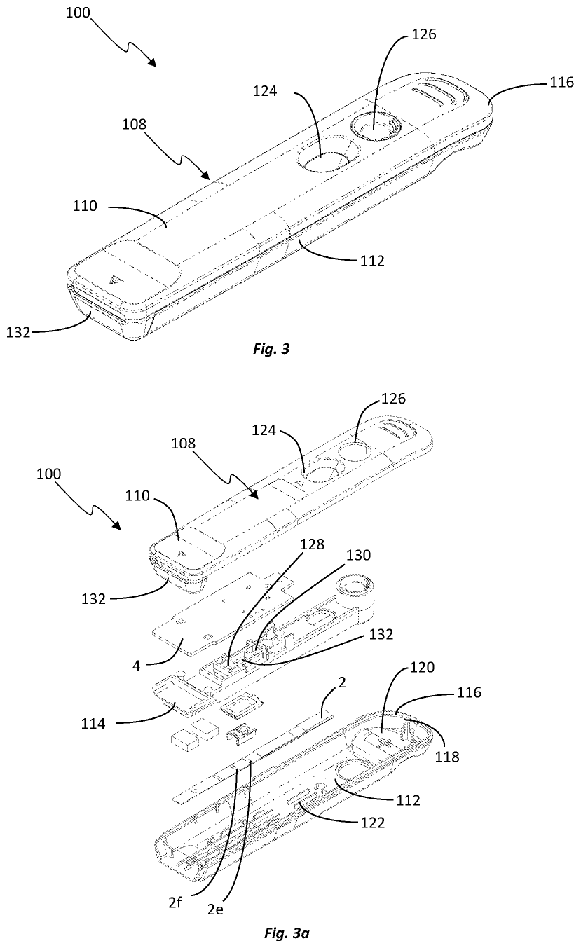

The test device 100 will now be described in further detail with reference to FIGS. 3 and 3a. The test device 100 comprises a housing 108 including upper and lower casings 110, 112 and an elongate base 114 located between the upper and lower casings 110, 112. As illustrated in FIG. 3a, the housing 108 defines an interior region in which an immunochromatographic (lateral flow) test strip 2 is located, providing an assay unit 102 of the test device 100, and also in which electronic circuitry, a processor and an optical assembly are located, providing a reader 104 of the test device 100.

Adjacent a first end 116 of the housing 108, a protuberance 118 projects from an inner surface 120 of the lower casing 112 and is adapted to fit into a registration hole (not shown) of the upper casing 110 by way of a snap-fit engagement. Guide ribs 122 formed in the inner surface 120 of the lower casing 112 serve to register the position of the test strip 2, relative to the base 114 and other components of the test device 100.

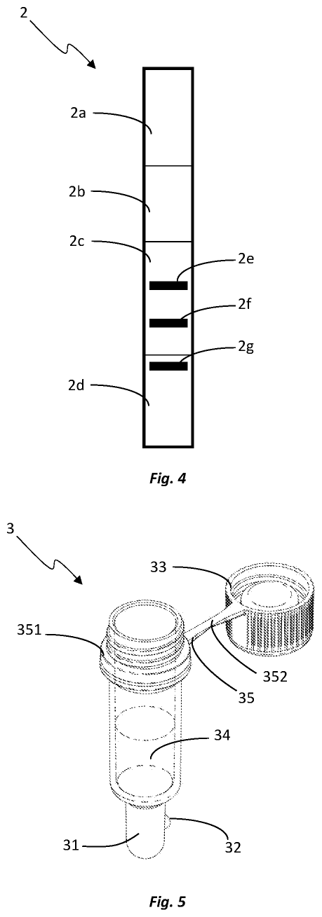

In this embodiment, the lateral flow test strip 2 includes several overlapping regions that are arranged sequentially along the length of the strip, as represented schematically in FIG. 4. The regions include a sample receiving region 2a, a label-holding region 2b, a test region 2c, and a sink 2d. The regions comprise chemically treated material such as chemically treated nitrocellulose, located on a waterproof layer. The design is such that a biological sample, when received and transferred from the sample receiving region 2a can transfer under capillary action into the label-holding region 2b, which contains a fluorescent substance for labelling target biological entities in the sample, and into the test region 2c where the sample will contact first and second test zones (first and second test stripes 2e, 2f in this embodiment) which each contain an immobilized compound capable of specifically binding the target biological entities or a complex that the target biological entities and the fluorescent labelling substance form. The sink (absorbent) region 2d is provided to capture any excess sample. Transfer of the sample along the test strip 2 can be assisted using a buffer solution, e.g. released from a fluid receptacle such as buffer bottle 3 as represented in FIG. 5. In some embodiments, the sample may be mixed with buffer solution, e.g. in the buffer bottle 3, prior to being applied to the test strip 2. In other embodiments, the sample may be applied to the test strip 2 separately from the buffer solution. The presence of the fluorescent labelled biological entity in the sample generally results in at least one of the test stripes 2e, 2f at the test region 2c being excitable by light in a particular wavelength band such as to cause a detectable level of florescent emission light to be emitted by the fluorescent label. A further control stripe 2g may also be provided to indicate that a testing procedure has been performed. The control stripe 2g can be located downstream of the first and second test stripes 2e, 2f to bind and retain the labelling substance. Detection of fluorescence at the control stripe 2g can indicate that sample has flowed through test zone 2c.

Depending on the degree of light detected at the test stripes 2e, 2f it may be determined that the target biological entity is present in the sample, and therefore a person providing the sample has a particular biological condition.

Referring again to FIGS. 3 and 3a, in this embodiment, the upper casing 110 includes a sample port 124 for receiving a biological sample. The sample port 124 is substantially of a funnel-like shape so as to assist with controlled delivery of the sample onto the test strip 2.

The upper casing 110 further includes an engagement element 126 for supporting and retaining the buffer bottle 3, or another type of fluid receptacle, used in conjunction with the test device 100. In this embodiment, the engagement element is a recess 126 that receives a first end, specifically a lower end 31, of the buffer bottle 3. The buffer bottle 3 is locked to the test device 100 by inserting the lower end 31 of the buffer bottle 3 into the recess 126 and rotating the buffer bottle 3, e.g. in an anti-clockwise direction. The lower end 31 of the buffer bottle 3 and the recess 126 have complimentary bayonet fittings 32, for example, which achieves locking on rotation.

At a second, upper end of the buffer bottle 3, a cap 33 is provided that is releasably securable to the buffer bottle 3, over an opening, via a screwthread. Buffer solution 34 may be deposited into the buffer bottle 3 via the opening and the cap 33 may be secured thereon, e.g. during manufacture or subsequently. Upon release of the cap 33, the cap 33 may remain connected to the buffer bottle 3 using a hinged arm 35. In this embodiment, the hinged arm 35 includes a collar portion 351 that fits around a rim of the opening of the buffer bottle 3, and a bendable limb 352 extending between the collar portion 351 and the cap 33 (in FIG. 5 the collar portion 351 is shown disconnected from the rim for simplicity).

In use, the buffer bottle 3, containing buffer solution 34, is locked to the test device 100 and the cap 33 subsequently released. A sample swab, or other sample-holding device, is inserted into the buffer bottle 3, via the opening, and optionally stirred within the buffer bottle 3, enabling mixing of the sample with the buffer solution 34. Once mixing is complete, the cap 33 may be replaced on top of the buffer bottle 3.

In this embodiment, to release the cap 33, the cap 33 is rotated in a direction, e.g., an anticlockwise direction, corresponding to a direction of rotation that is used to lock the buffer bottle 3 to the test device 100, ensuring that release of the cap 33 does not cause unintentional release of the buffer bottle 3 from the test device 100.

After mixing, the mixture of sample and buffer solution 34 may be held in the buffer bottle 3 and/or on the sample swab. The mixture may be applied from any one of these items to the test strip 2, via the sample port 124.

By providing an engagement element 126 for supporting and retaining the buffer bottle 3, e.g. during mixing with a buffer solution, manual operation of the system 10, including the base device 200 and test device 100, may be more straightforward. For example, a user may support the test device 100 in one hand, with the buffer bottle 3 projecting therefrom or a user may support the base device 200 in one hand, with the test device 100 and buffer bottle 3 projecting therefrom. Meanwhile, with their other hand, the user may remove the cap 33 of the buffer bottle 3, place the swab in the bottle 3, stir the swab, replace the cap 33 and apply the mixture to the sample port 124.

In a variation of the above, the sampling receiving port and the engagement element for receiving the buffer bottle may be combined. For example, the lower end 31 of the buffer bottle 3 may be locked into a bayonet fitting provided around the rim of the sample receiving port. In this variation, once a sample is mixed with the buffer solution 34 in the buffer bottle 3, the resulting mixture may be released directly from the locked-in buffer bottle 3 to the test strip 2, e.g. through a releasably sealed opening that may be included at the lower end 31 of the buffer bottle.

In this embodiment, the test device 100 is configured to test for the presence or absence of biological entities, for example influenza nucleoproteins, in a biological sample through fluorescence labelling, substantially in accordance with the fluorescence detection test discussed in PCT Publication No. WO 2014/201520, the content of which is incorporated herein by reference. For example, the first and/or second test stripes 2e, 2f of the test strip 2 are configured to bind fluorescent-labelled influenza nucleoproteins, if influenza nucleoproteins are present in the sample.

The reader 104 comprises one or more light sources and one or more light detectors. In this embodiment, the one or more light sources comprises a first LED 128 and a second LED 130, which are adapted to transmit excitation light to the test stripes 2e, 2f. The one or more light detectors may comprise a photodetector 132 positioned between the LEDs 128, 130 and which is configured to receive fluorescent emission light from the test stripes 2e, 2f. The amount of light reflected off the test stripes 2e, 2f is dependent on, e.g., the amount of fluorescent-labelled influenza nucleoproteins bound at the test stripes 2e, 2f. The optical assembly includes light guides (not shown) that are configured to guide excitation light from the LEDs 128, 130 to respective test stripes 2e, 2f, as well as to perform spectral filtering and focus, collimate and/or diverge light.

In this embodiment, the reader 104 also includes a printed circuit board (PCB) 4 connected to a processor and connectable to a power source. In combination with the photodetector, the processor is adapted to make a determination about the presence of influenza nucleoproteins in the biological sample based on the intensity of the light detected by the photodetector, as will now be described by way of example only.

In this embodiment, the PCB 4 may comprise a light-to-frequency converter that converts the light detected by the photodetector to a frequency. The light-to frequency converter may be a monolithic complementary metal-oxide-semiconductor (CMOS) integrated circuit, for example, which comprises a silicon photodiode and a current-to-frequency (CTF) converter. Infrared photons from the emission light promotes excited electrons to the silicon photodiode. The excited electrons undergo non-radiative relaxation to low-energy states and a corresponding photocurrent is induced. The photocurrent is proportional to the radiant flux incident on the photodetector. The photocurrent may then transmitted to the CTF converter to charge an associated capacitor. A voltage comparator, more particularly, in this embodiment an operational amplifier (op-amp), compares the voltage across the capacitor with a pre-defined threshold voltage. The PCB 4 is configured such that each time a pre-defined voltage threshold is reached across the terminals of the capacitor, the capacitor is drained and the state of the CTF converter output is changed from HIGH to LOW, for example. Once the capacitor is drained, an interrupt is sent to the processor and the CTF converter output state changes from LOW to HIGH, and the above process is repeated.

The processor is configured to determine the frequency of change of the CTF converter output state, based on whether the pre-defined voltage threshold is reached. The processor then determines the presence or absence of the fluorescent-labelled influenza nucleoproteins in the sample based on the frequency. The present disclosure recognises that the time taken to charge the capacitor is inversely proportional to the magnitude of the photocurrent. In this regard, the larger the radiant flux (i.e. fluorescent emission light) on the photodetector, the higher the frequency of change of the CTF converter output state.

The processor is adapted to determine whether a person providing the sample has, or does not have, a biological condition, more particular, in this embodiment influenza, based on the presence or absence of influenza nucleoproteins. The processor is also configured to generate a result data signal, including diagnostic data containing information about the determination of the biological condition.

The processor is also configured to conduct self-testing to determine whether components of the test device 100 are properly functioning. For example, the processor may be configured to test for adequate power supply across the electronic circuitry of the test device 100. The processor may also configured to generate status data containing information about the self-testing.

The test device connector 106 is located substantially adjacent a second end 132 of the housing 108 and is configured to link the test device 100 to the electronics unit 202 of the base device 200 so as to allow for data transmission between the test device 100 and the electronics unit 202 and to receive power from the electronics unit 202. In this embodiment, the test device connector 106 may comprise a set of contacts, for example. The set of contacts may be a separate or integrated component of the PCB 4. It will be appreciated, however, that the test device connector 106 of the present disclosure may take a variety of other forms.

The test device 100 according to any of the above embodiments may be a hand-held device. Accordingly, the test device 100 may be differentiated in this regard from an apparatus that could employ an electronic reader in a laboratory environment to analyse a test portion. The test device 100 may also be disposable, configured for single-use only.

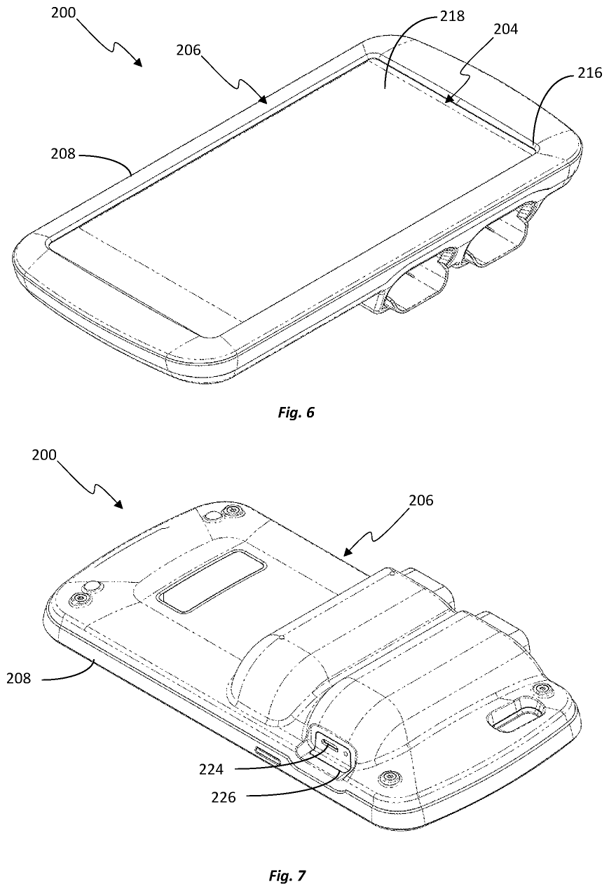

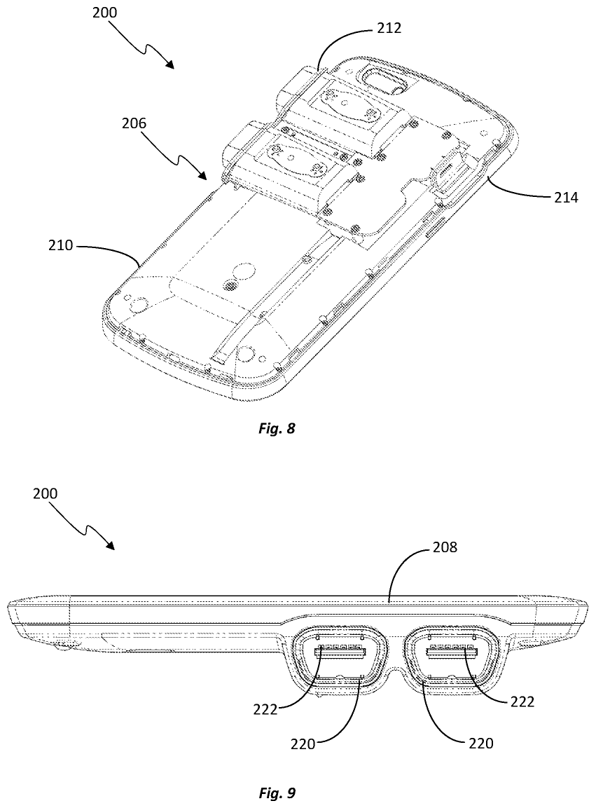

The base device 200 will now be described in further detail with reference to FIGS. 6 to 9. The base device 200 comprises an electronics unit 202 and a display unit 204. The electronics unit 202 comprises a housing 206 including an outer casing 208, a base 210 and a connection element 212 located within the outer casing 208. The housing 206 defines an interior region in which a power source and electronic circuitry are located.

The base 210 of the housing 206 includes a front wall (not shown) and side walls 214 projecting from the edges of the front wall. The inner surface of the front wall and the inner surfaces of the side walls 214 together define a recessed portion of the base 210 that is adapted to at least partially house the display unit 204. The outer casing 208 includes a window 216 through which a display 218 of the display unit 204 can be observed by a user, as illustrated in FIG. 6. In this embodiment, the outer casing 208 substantially encases the display unit 204 except for the display 218 of the display unit 204.

Referring to FIG. 9, the connection element 212 includes one or more ports 220 for releasably receiving one or more respective test devices 100. The ports 220 are provided at a side of the base device 200. In some embodiments, each port 220 may be in the form of a plug-in interface, for example. The ports 220 are each configured to receive the second end 132 of the test device 100 and more particularly to electrically couple to the test device connector 106 of the test device 100. In this embodiment, the ports 220 each includes a set of contacts 222 configured to contact with the set of contacts of the test device connector 106. In some embodiments, the set of contacts 222 are provided by spring contacts that may compress upon contact with the set of contacts of the test device connector 106 when the test device 100 is inserted in the port 220. It will be appreciated, however, that the ports 220 may take a variety of other forms, so long as the ports 220 and the test device connector 106 are each configured to be operatively associated with one another. The connection element 212 is configured to allow for data transmission between the electronics unit 202 and the test device 100 and for power transmission to the test device 100.

The electronic circuitry of the electronics unit 202 includes a first PCB connected to the power source, the connection element 212 and a processor. The first PCB includes connectivity components for detecting successful connection between the test device 100 and the electronics unit 202. In this embodiment, the connectivity components may include a connectivity resistor operatively associated with one of the spring contacts 222 of the connection element 220. The connectivity resistor may be a pull-up resistor, for example, nominally pulled high by a large value resistor. The test device 100 may also comprise a corresponding connectivity resistor operatively associated with one of the contacts of the test device connector 106. Upon successful connection of the test device connector 106 to the electronics unit 202, the connectivity resistor of the electronics unit 202 is pulled low by the connectivity resistor of the test device 100, resulting in a voltage drop across the two connectivity resistors. The processor of the base device 200 is configured to register the voltage drop as a successful connection between the test device 100 and the electronics unit 202. In this embodiment, data and/or power transmission between the electronics unit 202 and the test device 100 only commences after a successful connection has been registered.

In this embodiment, the electronic circuitry of the electronics unit may also include a second PCB coupled to the first PCB, and a connector that links the display unit 204 to the second PCB. The connector may be adapted to connect to an input/output port of the display unit 204 so as to allow for data and/or power transmission between the display unit 204 and the electronics unit 202. In some embodiments, the connector may be provided as a standard microUSB connector and the input port of the display unit 204 may be provided as a standard microUSB port.

In this embodiment, electronic circuitry of the base device 200 may also include a recharging unit for receiving power from an external power source. The recharging unit may include a recharging port 224 located on the side of the base device 200 for releasably receiving the external power source. The recharging port 224 may be accessible through an opening 226 in the outer casing 208, as illustrated in FIG. 7. In some embodiments, the recharging port 224 may be provided as a microUSB port or otherwise.

The first PCB may also be configured to distribute power between (1) the electronics unit 202 and the test device 100, and (2) the electronics unit 202 and the display unit 204. In this embodiment, power may be transmitted from the power source of the electronics unit 202 to the power source of the test device 100 upon successful connection of the test device 100 to the electronics unit 202. A series of conditioning elements may be provided to regulate the transmission of power to the test device 100 as well as to remove noise from power supply routes. The conditioning elements can prevent damage (e.g. power surge) to the electronics unit 202 and/or the test device 100 by way of an incompatible connection of an external power supply to the recharging port of the electronics unit 202, for example. In some embodiments, the test device 100 may have no integral power source, with all power, e.g. for the performing of the reader operations, being provided from the electronics unit 202.

In this embodiment, power may also be transmitted from the power source of the electronics unit 202 to the display unit 204 via the second PCB.

The first PCB also includes components for data transmission between the test device 100 and the display unit 204. For example, in this embodiment, the first PCB may be provided with a USB-to-serial dual channel bridge integrated circuit in conjunction with a universal asynchronous receiver/transmitter (UART). The UART may be configured to receive the result data signal from the test device 100 and translate the data to a format compatible with USB serial communications. A data transfer protocol may be utilised to determine the origin of the received result data signal (e.g. test device 1, test device 2) as well as to identify the information contained in the data (i.e. information from diagnostic analysis or information from self-testing). The received result data signal is transmitted to the display unit 204, via the second PCB.

In the present embodiment, the display unit 204 is manufactured separately from the electronics unit 202 and the display unit 204 and electronics unit 202 are connected together prior to use. The display unit may be provided by a computer device, such as a smartphone, personal digital assistant, tablet computer or otherwise. The display unit 204 may also include a graphical user interface (GUI) configured to allow users to present the result data signal from the test device 100 on the display 218 of the display unit 200.

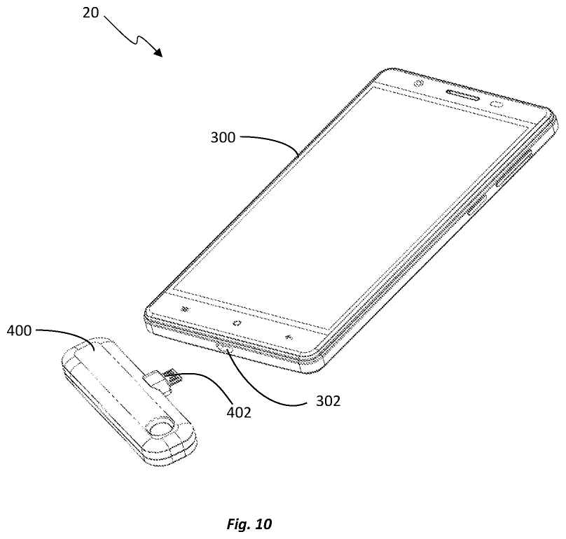

FIG. 10 shows a further application of the embodiments of the present disclosure. In the diagnostic system 20 of this embodiment, the display unit is integrated with the electronics unit during manufacture. The entire base device 300 may be provided by a computer device, such as a smartphone, personal digital assistant, tablet computer or otherwise. The base device 300 may include a port 302 for releasably receiving the test device 400 and specifically to electrically couple to the test device 400 so as to allow for data and/or power transmission between the base device 300 and the test device 400. In some embodiments, the port 302 may be a standard input/output port of the base device 300, such as a microUSB port, for example.

The test device 400 is similar to that shown in FIGS. 3 and 3a, and may include an assay unit, a reader and a test device connector 402. In this embodiment, however, the test device connector 402 may be provided as a standard connector operatively associated with the standard input/output port 302 of the base device 300. The test device 400 may be configured to receive a biological sample and perform diagnostic analysis on the sample to generate a result data signal for sending to the base device 300, similar to the embodiments described above.

The base device 200, 300 according to any of the above embodiments may be a hand-held device. However, in alternative embodiments, the base device may be arranged to rest on a desk, table or other surface.

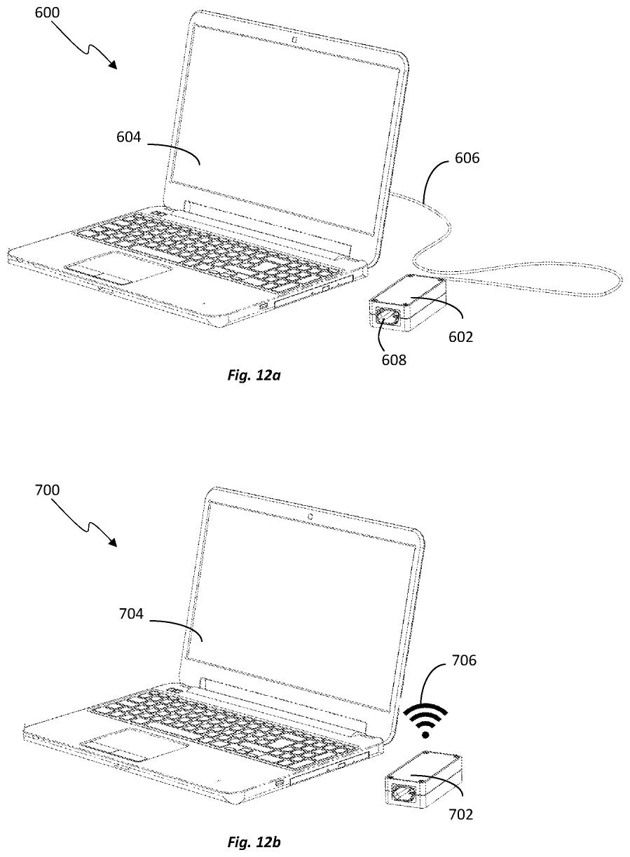

In one embodiment, as shown in FIG. 12a, a base device 600 comprises an electronics unit 602 and comprises, or is at least configured to connect to, a separate display unit 604 that is connected to the electronics unit 602 via a cable 606. In this embodiment, the display unit 604 is provided by a laptop computer 604. The cable 606 connects the electronics unit 602 to a port of the computer 604, e.g., a USB port or otherwise.

The base device 600 includes a single port 608 to releasably receive and connect to the test device, such as test device 100, in a similar manner to that described above with reference to FIGS. 3 and 3a. Like preceding embodiments, the electronics unit 602 is configured to distribute power to the test device, either from a power source such as a rechargeable battery that is comprised in the electronics unit 604 and/or from a power source such as a rechargeable battery that is comprised in the display unit 604. Moreover, the electronics unit 602 is configured to receive the result data signal from the test device 100 and communicate with the display unit 604 for displaying results of testing. For example, the electronics unit 602 may include a display controller that controls the display unit 604 to present results of testing on its display and/or the display unit 604 may include a display controller that controls the presentation of the results of testing on its display.

In an alternative embodiment, as shown in FIG. 12b, a base device 700 is provided that comprises an electronics unit 702 and comprises, or is at least configured to connect to, a separate display unit 704, generally accordance with the base device 600 described above, except that communication between the electronics unit 702 and the display unit 704 is via a wireless link 706 such as a Bluetooth link, WiFi link or otherwise, rather than via a cable. In this embodiment, the electronics unit 702 is configured to distribute power to the test device from a power source, such as a rechargeable battery, that is comprised in the electronics unit 702.

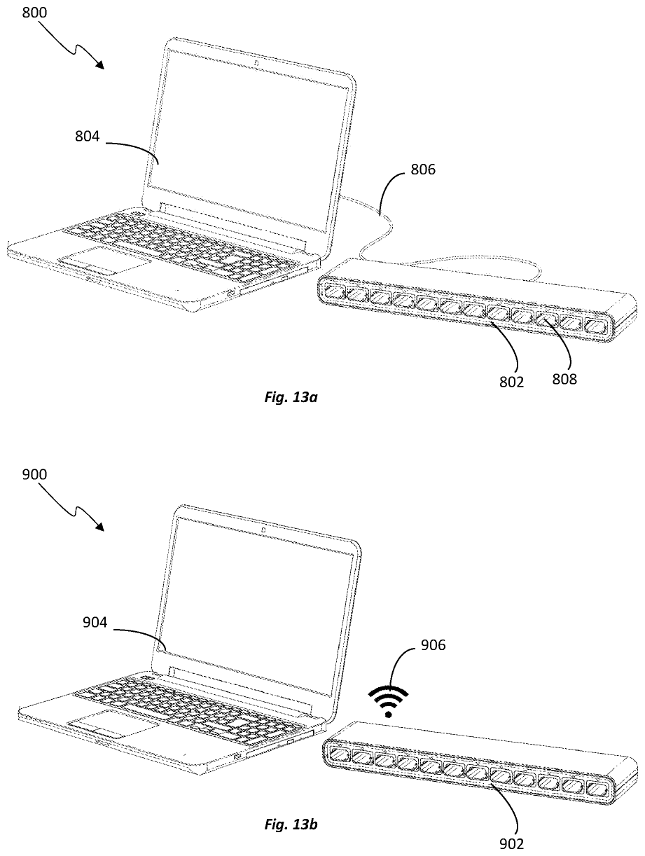

In yet an alternative embodiment, as shown in FIG. 13a, a base device 800 is provided that comprises an electronics unit 802 and comprises, or is at least configured to connect to, a separate display unit 804, generally accordance with the base device 600 described above, except that the electronics unit includes multiple ports 808 to releasably receive respective test devices, such as a test devices 100 as described above with reference to FIGS. 3 and 3a. Diagnostic analysis of the sample can again be carried out solely by each test device 100, significantly reducing processing requirements of the base device 800 and allowing for multiple test devices 100 to be connected simultaneously to the electronics unit 802 of the base device 800 without requiring any bespoke reader elements in the base device 800.

In an alternative embodiment, as shown in FIG. 13b, a base device 900 is provided that comprises an electronics unit 902 and comprises, or is at least configured to connect to, a separate display unit 904, generally accordance with the base device 800 described above, except that communication between the electronics unit 902 and the display unit 904 is via a wireless link 906 such as a Bluetooth link, WiFi link or otherwise, rather than via a cable. In this embodiment, the electronics unit 902 is configured to distribute power to the test device from a power source, such as a rechargeable battery, that is comprised in the electronics unit 902.

Generally, it will be recognised that the processor used with the diagnostic system according to any of the embodiments of the present disclosure can comprise a number of control or processing modules for controlling one or more components of the system and may also include one or more storage elements, for storing data. The modules and storage elements can be implemented using one or more processing devices and one or more data storage units, which modules and/or storage devices may be at one location or distributed across multiple locations and interconnected by one or more communication links.

Further, the modules can be implemented by a computer program or program code comprising program instructions. The computer program instructions can include source code, object code, machine code or any other stored data that is operable to cause the controller to perform the steps described. The computer program can be written in any form of programming language, including compiled or interpreted languages and can be deployed in any form, including as a stand-alone program or as a module, component, subroutine or other unit suitable for use in a computing environment. The data storage device(s) may include a non-transitory computer-readable memory medium comprising instructions that cause the processor to perform steps as described herein. The data storage device(s) may include suitable computer readable media such as volatile (e.g., RAM) and/or non-volatile (e.g., ROM, disk) memory or otherwise.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A diagnostic system comprising:
at least one test device for performing diagnostic analysis of a biological sample from a human or animal body, the at least one test device comprising:
an assay unit configured to receive the biological sample for testing for the presence or absence of one or more biological entities in the biological sample; and
a reader configured to determine one or more biological conditions based on the presence or absence of the one or more biological entities at the assay unit, and to generate a result data signal containing information on at least the determination of the one or more biological conditions; and
a base device comprising a port for releasably receiving the test device; and a receiver for receiving the result data signal when the test device is received by the base device; the base device further comprising, or being configured to connect to, a display for displaying results of testing of the biological sample based on the received result data signal.

2. The system of claim 1, wherein the one or more biological entities provides a marker for the one or more biological conditions.

3. The system of claim 1 or 2, wherein the one or more biological conditions comprises one or more of chlamydia, colorectal cancer, Helicobacter pylori infection, influenza, legionella, mononucleosis, ovulation phase in a menstrual phase, osteoporosis, pregnancy, prior subjection to myocardial infarction, renal failure, respiratory syncytial virus infection, pneumococcal infection, and streptococcal pharyngitis.

4. The system of any one of the preceding claims, wherein the assay unit comprises a test portion to test for the presence of absence of a biological entity in the biological sample.

5. The system of claim 4, wherein the test portion is an immunochromatography test strip.

6. The system of claim 4 or 5, wherein the reader comprises one or more light sources adapted to transmit excitation light to one or more portions of the test strip, and one or more light detectors capable of monitoring light reflection or light output at one or more portions of the test strip.

7. The system of claim 6, wherein the reader comprises a processor configured to make a determination about the presence or absence of the one or more biological entities in the biological sample based on the intensity of the light detected by the one or more light detectors.

8. The system of any one of the preceding claims, wherein the base device comprises an electronics unit that includes the port to receive the test device, the electronics unit being configured to electrically couple to the test device so as to allow for data transmission between the test device and the electronics unit.

9. The system of claim 8, wherein the electronics unit comprises connectivity components for detecting a successful connection between the at least one test device and the electronics unit.

10. The system of claim 8 or 9, wherein the base device comprises a display unit that includes the display.

11. The system of claim 10, wherein the display unit is integrated with the electronics unit during manufacture.

12. The system of claim 11, wherein the base device is a smartphone, personal digital assistant or tablet computer.

13. The system of claim 10, wherein the display unit is manufactured separately from the electronics unit and is connected to the electronics unit prior to use.

14. The system of claim 13, wherein the display unit is a smartphone, personal digital assistant or tablet computer.

15. The system of claim 13 or 14, wherein the electronics unit comprises a housing adapted to at least partially house the display unit.

16. The system of claim 15, wherein the electronics unit comprises a window or opening through which the display of the display unit can be observed.

17. The system of any one of claims 8 to 16, wherein the electronics unit is configured to distribute power to the at least one test device and/or the display unit.

18. The system of claim 17, wherein the electronics unit comprises a series of conditioning elements to regulate the transmission of power to the test device and/or the display unit.

19. The system of claim 17 or 18, wherein the at least one test device has no integral power source.

20. The system of any one of the preceding claims, wherein the base device includes two or more ports, each of the two or more ports being configured to releasably receive a test device.

21. The system of any one of the preceding claims, wherein the base device is provided in the form of a hand-held device.

22. The system of any one of the preceding claims, wherein at least one test device is a hand-held device.

23. The system of any one of the preceding claims, wherein at least one test device is disposable and configured for single-use only.

24. A test device for a base device having a port for releasably receiving the test device, the test device comprising:
an assay unit configured to receive a biological sample from a human or animal body for testing for the presence or absence of one or more biological entities in the biological sample; and
a reader configured to determine one or more biological conditions based on the presence or absence of the one or more biological entities at the assay unit, and to generate a result data signal containing information on at least the determination of the one or more biological conditions,
wherein the base device is configured to receive the result data signal when the test device is received by the base device for displaying results of testing of the biological sample based on the received result data signal.

25. The device of claim 24, wherein the one or more biological entities provides a marker for the one or more biological conditions.

26. The device of claim 24 or 25, wherein the one or more biological conditions comprises one or more of chlamydia, colorectal cancer, Helicobacter pylori infection, influenza, legionella, mononucleosis, ovulation phase in a menstrual phase, osteoporosis, pregnancy, prior subjection to myocardial infarction, renal failure, respiratory syncytial virus infection, pneumococcal infection, and streptococcal pharyngitis.

27. The device of any one of claims 24 to 26, wherein the assay unit comprises a test portion to test for the presence of absence of a biological entity in the biological sample.

28. The device of claim 27, wherein the test portion is an immunochromatography test strip.

29. The device of claim 27 or 28, wherein the reader comprises one or more light sources adapted to transmit excitation light to one or more portions of the test strip, and one or more light detectors capable of monitoring light reflection or light output at one or more portions of the test strip.

30. The device of claim 29, wherein the reader comprises a processor configured to make a determination about the presence or absence of the one or more biological entities in the biological sample based on the intensity of the light detected by the one or more light detectors.

31. A method for performing diagnostic analysis of a biological sample from a human or animal body, the method comprising:
receiving the biological sample in a test device;
testing, using the test device, for the presence or absence of one or more biological entities in the biological sample;
determining, using the test device, one or more biological conditions based on the presence or absence of the one or more biological entities in the biological sample;
generating, at the test device, a result data signal containing information on at least the determination of the one or more biological conditions;
connecting the test device to a port of the base device;
receiving the result data signal by a receiver of the base device when the test device is connected to the base device; and
displaying results of testing of the biological sample based on the received result data signal.

32. The method of claim 31, wherein at least the steps of determining and generating are carried out by the test device when the test device is connected to the base device.

33. The method of claim 32 comprising distributing power from the base device to the test device when the test device is connected to the base device, to power the test device for performing at least the steps of determining and/or generating.

34. A test device for testing for the presence or absence of one or more biological entities in a biological sample, the test device comprising a housing, the housing having an engagement element for releasably retaining a fluid receptacle on an outer surface of the housing.

35. The test device of claim 34, wherein the engagement element is a recess adapted to retain a first end of the fluid receptacle.

36. The test device of claim 34 or 35, wherein the engagement element is adapted to receive the fluid receptacle in a releasably-locked manner.

37. The test device of claim 36, wherein the engagement element has a bayonet fitting for releasably locking to a complimentary bayonet fitting of the fluid receptacle.

38. The test device of any one of claims 34 to 37, wherein the fluid receptacle comprises buffer solution.

39. A fluid receptacle configured to be releasably retained to a test device, the test device comprising a housing, the housing having an engagement element for releasably retaining the fluid receptacle on an outer surface of the housing.

40. The fluid receptacle of claim 39, wherein the engagement element is a recess and a first end of the fluid receptacle is adapted to be retained in the recess.

41. The fluid receptacle of claim 39 or 40, wherein the first end is adapted to be retained in the recess in a releasably-locked manner.

42. The fluid receptacle of claim 41, wherein the fluid receptacle has a bayonet fitting for releasably locking to a complimentary bayonet fitting of the engagement element.

43. The fluid receptacle of any one of claims 39 to 42, wherein the fluid receptacle comprises buffer solution.

44. The fluid receptacle of any one of claims 39 to 43, wherein the fluid receptacle comprises a cap.
